(19) United States
(12) Patent Application Publication
(10) Pub. No.: US 2023/0194390 A1
(43) Pub. Date: Jun. 22, 2023

(54) AUTOMATIC SAMPLING APPARATUS AND METHOD FOR AUTOMATICALLY PROVIDING A SAMPLE FOR QUALITATIVE AND/OR QUANTITATIVE DETERMINATION OF AN ANALYTE

(71) Applicant: Endress+Hauser Conducta GmbH+Co. KG, Gerlingen (DE)

(72) Inventors: Urs Endress, Arlesheim (CH); Detlev Wittmer, Maulbronn (DE); Manfred Jagiella, Notzingen (DE); Christoph Rompf, Stuttgart (DE); Ralf Steuerwald, Eberdingen (DE); Angela Eubisch, Chemnitz (DE); Michael Hanko, Dresden (DE); Peter Lindmüller, Essingen (DE); Eckhard Sommer, Sternenfels (DE); Robert Möller, Jena (DE); Christine Marion Gräfe, Jena (DE); Monika Heisterkamp, Ditzingen (DE)

(21) Appl. No.: 17/998,847

(22) PCT Filed: Apr. 19, 2021
(86) PCT No.: PCT/EP2021/060101

§ 371 Date: Nov. 15, 2022

(30) Foreign Application Priority Data
May 15, 2021 (DE) 10 2021 113 317.0

(51) Int. Cl. G01N 1/14; G01N 35/10

(52) U.S. Cl. CPC G01N 1/14; G01N 35/1016; G01N 2001/1418; G01N 35/0098

(57) ABSTRACT

An automatic sampling apparatus for taking liquid samples for the qualitative or quantitative determination of at least one analyte contained in the sample liquid includes a sample line that can be fluidically connected to the sampling station, a pump device, at least one sample container that can be fluidically connected to the sample line, and an electronic control system. The electronic control system is configured to fluidically connect the sample line to the sample container such that a fluid flow path extending from the sampling station through the sample line into the sample container is formed. The electronic control system is further configured to transport, using the pump device, a definable volume of the sample liquid, in the form of a liquid sample, along the fluid flow path into the sample container. The sampling apparatus is configured for concentrating or extracting the analyte present in the liquid sample.

The invention relates to an automatic sampling apparatus and a method for automatically providing a sample for qualitative and/or quantitative determination of an analyte comprising a biopolymer in a sample liquid, in particular in water or wastewater. The invention also relates to a measurement system for the qualitative or quantitative determination of the analyte, which comprises the automatic sampling apparatus and a method for the qualitative or quantitative determination of the analyte in the sample liquid.

The analyte can be a bioparticle and/or a biopolymer, e.g., DNA, RNA, a protein, a virus, a virus component, a bacterium or a bacterial component, or an anthropogenic trace element or micropollutant, e.g., a pesticide, drug, body care product, or degradation products or metabolites thereof.

It is known from S. Mallapaty, “How sewage could reveal true scale of coronavirus outbreak,” Nature, Vol. 580, 2020, p. 176, that virus components, e.g., RNA, can be detected in the municipal wastewater of a region in which virus infections currently occur. The verification of such virus components is performed by manually taking water samples from a sewage plant and subsequently examining them in the laboratory. Generally, the laboratory examination initially comprises complex preparatory methods for concentrating and/or extracting the analyte in order to provide a sample suitable for the qualitative or quantitative analysis. The sample produced in this way is subsequently analyzed by means of an immunological method or a method based upon the real-time quantitative PCR, or qPCR for short. This procedure is not suitable for the systematic monitoring of wastewater, which could also serve as a basis for an early warning system for extended geographical areas, e.g., for a large number of municipalities or districts, since the time and personnel effort associated with this is very high.

The object of the invention is to provide an apparatus and a method which allow systematic monitoring of water or wastewater for the occurrence of certain analytes, e.g., biopolymers or bioparticles, such as viruses, virus components, bacteria, bacterial components, proteins, DNA or RNA, or anthropogenic micropollutants.

The object is achieved by the automatic sampling apparatus according to claim 1, the measurement system according to claim 12, the method for automatically providing a sample according to claim 16, and the method for the qualitative or quantitative determination of at least one analyte according to claim 23. Advantageous embodiments are given in the dependent claims.

The automatic sampling apparatus according to the invention for taking liquid samples of a sample liquid, in particular water or wastewater, present at a sampling station of a body of water or of a container, for the qualitative or quantitative determination of at least one analyte, in particular a biopolymer, contained in the sample liquid, comprises: a sample line that can be fluidically connected to the sampling station;

a pump device; at least one sample container that can be fluidically connected to the sample line; and an electronic control system which is configured to fluidically connect the sample line to the at least one sample container such that a fluid flow path extending from the sampling station through the sample line into the sample container is formed, and wherein the electronic control system is further configured to transport, by means of the pump device, a definable volume of the sample liquid, in the form of a liquid sample, along the fluid flow path into the sample container, wherein the sampling apparatus has means for concentrating and/or extracting the analyte present in the liquid sample.

By performing the concentration and/or extraction of the analyte present in the sample liquid by means of the sampling apparatus, a sample can be provided which is prepared for subsequent quantitative or qualitative determination, the volume of which sample is significantly reduced compared to the liquid sample originally taken from the sampling station.

This facilitates the transport of the sample to be examined to a laboratory for further analysis. The sample obtained by concentration and/or extraction is also, advantageously, suitable for transfer, in particular automatic transfer, to an analytical instrument. The taking of the liquid sample and the concentration or extraction can, advantageously, be carried out partially or completely automatically by means of the sampling apparatus. This allows the sample liquid to be systematically monitored for the presence of the analyte at an individual sampling station, or also at a plurality of sampling stations in an extended geographical area.

Qualitative determination of the analyte is understood here and below to mean a method in which it is determined whether the analyte is present in the liquid sample. A quantitative determination moreover comprises the determination of a value representing the concentration of the analyte in the liquid sample.

The apparatus can be configured to concentrate and/or extract one or more analytes to be determined. The at least one analyte can be a bioparticle or a biopolymer, e.g., DNA, RNA, a protein, a virus, a virus component, a bacterium or a bacterial component, or an anthropogenic trace element or micropollutant, e.g., a pesticide, drug, body care product, or the degradation product or metabolite thereof.

The sample liquid can be wastewater, water present in a body of water, or water, in particular drinking water, in a supply network. The sampling station mentioned can, for example, be a point on or in a body of water. A container containing the sample liquid can be a pipeline through which the sample liquid flows or an open basin or channel. Accordingly, the sampling station can also be arranged on or in a pipeline, which is fluidically connected or can be connected to the sample line and through which the sample liquid flows, e.g., in a water supply network or in a wastewater network, or on or in a basin - for example, in a local or municipal sewage treatment plant. The sampling station can, for example, be a point in the region of the inflow of a sewage treatment plant. It can also be arranged at a point of a wastewater network, e.g., in a discharge point or in the region of a discharge point into the wastewater network.

The sampling apparatus can have several sample containers, each of which can be fluidically connected to the sample line. For this purpose, the sampling apparatus can have a sample distributor fluidically connected to the sample line, e.g., in the form of a movable distributor arm, the position of which can be adjusted by the electronic control system in order to fluidically connect one of the sample containers in each case to the sample line by means of the sample distributor. Alternatively, the sample distributor can also be realized by means of a valve device and fluid lines, which can be individually connected via the valve device to the sample line and which lead to the sample containers.

Optionally, a filter device having a filter or a filter cascade can be arranged in the fluid flow path extending between the sampling station and the sample container, and preferably upstream of the sample container, which filter device is designed to filter solids out of the sample liquid, but allow the analyte to pass through.

The means for concentration and/or extraction may comprise a collection matrix which can be brought into contact with the liquid sample and which selectively or substantially selectively binds the analyte. The collection matrix can be designed, for example, to selectively bind or immobilize a specific class of biopolymers, e.g., certain proteins or RNA or DNA of specific species, such as, for example, RNA of viruses of a specific type, e.g., a specific order, family, or genus. If the analyte is an anthropogenic trace element, the collection matrix can be configured to selectively bind or immobilize a specific substance or a specific class of low-molecular-weight organic substances.

In one possible embodiment, the collection matrix can be arranged in the at least one sample container or in a flow-through unit fluidically connected to the sample container, which unit is in particular a fluid line or a cartridge through which liquid can flow. The flow-through unit can be arranged in the fluid flow path, and in particular upstream of the sample container. It can also be arranged in an upper region of the sample container - for example, as an insert close to or in an opening of the sample container. The flow-through unit can be detachably connected to the fluid line and/or the sample container so that it is possible to remove the flow-through unit together with the collection matrix and the analyte bound thereto from the sampling apparatus and from the sample container and transport it to a laboratory for further analysis. Alternatively, the collection matrix can be loose or in a removable insert in the sample container. In this case, the collection matrix with the analyte bound thereto can be removed by removing the insert or by emptying the sample container while retaining the collection matrix.

In order to concentrate and/or extract more than one analyte, the collection matrix can be designed to immobilize several different, but in particular structurally and/or chemically similar, analytes - for example, as already mentioned, specific proteins or a specific class of biopolymers. In a sample obtained by eluting the immobilized analytes from the collection matrix, all analytes can be determined, qualitatively or quantitatively, independently of one another - for example, by means of qPCR-based methods.

In order to concentrate and/or extract more than one analyte, it is also possible to provide several different collection matrices in the sample container or in a flow-through unit fluidically connected to the sample container. For example, the sampling apparatus can have several through-flow cartridges, which are arranged in the fluid flow path or in the sample container and which each contain different collection matrices which are each designed for the immobilization of at least one specific analyte. The sampling apparatus can also have several sample containers, each containing different collection matrices such that each collection matrix immobilizes another analyte. Different liquid samples thus serve to determine different analytes.

The collection matrix advantageously has a large surface area. The surface can have a functionalization which serves to selectively bind the analyte, e.g., RNA, DNA, or proteins. For this purpose, said surface can be coated with silica, glass, or crosslinked polymers which are modified for selective interaction with the analyte (e.g., positively-charged viruses) with suitable functional groups (e.g., negatively-charged hydroxy groups). It can also consist entirely of silica, glass, or cross-linked polymers which are modified for selective interaction with the analyte. It is also possible for the surface to be modified by antibodies, interacting specifically with the analyte, or similar capture structures or capture molecules or identification elements which are based upon affine interactions. Examples of capture structures or identification elements include aptamers, peptide nucleic acids (PNA), and molecular imprinted polymers (MIP).

The collection matrix can have, for example, a multiplicity of surface-functionalized particles (beads or microbeads), e.g., polymer beads or magnetic beads, a surface-functionalized substrate with a roughened or three-dimensionally structured surface, a functionalized nonwoven, e.g., a functionalized fleece filter, or a chromatographic separation column.

The automatic sampling apparatus can be configured to automatically empty and clean the at least one sample container before or after manual or automatic removal of the collection matrix with the bound analyte, so that the sample container is available for receiving a new liquid sample.

The sampling apparatus may further be configured to introduce a new collection matrix for selective binding or immobilization of the at least one analyte in the sample container after the sample container has been emptied and cleaned, and the collection matrix loaded with the analyte has been removed. Alternatively, the sampling apparatus can also be configured to automatically empty the sample container, to elute the analytes retained in the collection matrix from the collection matrix, and to collect the eluate as a sample provided for further analysis or to output it as a sample to an automatic analytical instrument connected to the sampling apparatus. In this embodiment, the collecting matrix can remain in the sample container. After removal of the eluate and an optional purification process, the sample container with the remaining collection matrix is again available for receiving a new liquid sample and for the concentration and extraction of the analyte in this liquid sample by means of the collection matrix.

In an alternative embodiment, the means for concentration and/or extraction can comprise a centrifuge integrated into the apparatus.

The electronic control system can be configured to store an identifier of the liquid sample for each liquid sample taken from the sampling station, and to save a time stamp, indicating the time at which the liquid sample is taken from the sampling station, to the identifier. The identifier of the liquid sample can simultaneously be an identifier of the sample container into which the liquid sample is transported. The electronic control system can be configured to store the identifier and the time stamp locally in a memory of the electronic control system and/or to communicate to a device connected to the electronic control system for communication, e.g., a portable operating device (e.g., smartphone, tablet), a PC connected to the electronic control system for communication, or a server. The server can be a component of the sampling apparatus, in particular a component of the electronic control system, or a computer that is remote from the sampling apparatus, and in particular part of a cloud.

The automatic sampling apparatus can further have at least one interface for connecting the electronic control system to at least one sensor which can be brought into contact with the sample liquid present at the sampling station, and which is designed to generate measurement signals of at least one measurand of the sample liquid, e.g., temperature, pH, or conductivity, and output said measurement signals to the electronic control system, wherein the electronic control system is further configured to save at least one measured value, derived from the measurement signals, of the measurand, which is acquired at the time at which the liquid sample is taken from the sampling station, to the identifier of the liquid sample. By means of the additionally acquired measurand, a measured value, determined on the basis of the liquid sample, of the analyte concentration in the sample liquid can be calculated more precisely. The electronic control system can be configured to locally store the measured values and/or to communicate to the aforementioned device connected to the electronic control system for communication.

Further measurands, which can be measured by means of one or more additional sensors that can be connected to the electronic control system, are turbidity, concentrations of dissolved gases, ion concentrations, or concentrations of photometrically or spectrometrically detectable molecules or cumulative parameters, such as SAC value (spectral absorption coefficient), COD (chemical oxygen demand), or TOC (total organically-bound carbon).

The automatic sampling apparatus may optionally comprise means for adjusting a pH in the liquid sample. These means can comprise, for example, a pump controllable by means of the electronic control system, optionally additional liquid lines, and one or more reagents, e.g., one or more buffer solutions, one or more acids, and/or one or more bases, which can be transported by means of the pump via the fluid lines into the sample container in order to adjust the pH value of the liquid sample contained therein. Optionally, a pH sensor can be arranged in the sample container, which is connected to the electronic control system in order to output said measured pH values. Based upon these pH measurements, the electronic control system can regulate the pH in the liquid sample. Alternatively, the electronic control system can be connected, via the interface mentioned in the previous paragraph, to a pH sensor, which is immersed in the sample liquid at the sampling station, and, based upon the measured pH value acquired by said pH sensor at the time the liquid sample was taken, can add one or more of the reagents to the liquid sample in the sample container in order to adjust the pH. This is advantageous in systems in which the analyte is concentrated by selective adsorption on a collection matrix, wherein the adsorption preferably takes place in a certain pH range by means of electrostatic interactions.

In a further embodiment, the sampling apparatus can have a temperature sensor, which is designed to acquire measured values representing a temperature of a liquid sample contained in the at least one sample container, and to output these values to the electronic control system.

The electronic control system can further be configured to save at least one of the measured values or a measured value profile to the identifier of the liquid sample. The electronic control system can be configured to locally store the measured values and/or to communicate to the device mentioned above and connected to the electronic control system for communication. In this way, a temperature or a temperature profile, to which the liquid sample with the analyte is exposed, can, until the collection matrix or a sample provided by concentrating and extracting the analyte from the liquid sample is removed from the sample container or from the sampling apparatus for the purpose of further analysis, be taken into account in the determination and/or interpretation of the analytical findings. For example, the temperature profile can be included in a determination of the analyte concentration in the sample liquid at the time of sampling.

For one thing, the temperature of the sample liquid can influence the speed of degradation processes of the analyte or analytes. On the other hand, it can simultaneously influence the binding kinetics of the analyte or analytes to the collection matrix. It is therefore advantageous to record the temperature or the temperature profile to which the liquid sample and/or the collection matrix is exposed after the liquid sample is taken from the sampling station, and to provide it for consideration during the qualitative and/or quantitative determination of the at least one analyte.

In a further embodiment, the automatic sampling apparatus can comprise a temperature control device which is designed to control the temperature of, and in particular to cool, the at least one sample container and the liquid sample contained therein. Optionally, the temperature control device can be configured to adjust a specific temperature in the region of the at least one sample container.

In a further embodiment, the sampling apparatus can have a GPS receiver which is configured to output location data (GPS data) to the electronic control system, wherein the electronic control system is configured to store the location data. The electronic control system can be configured to locally store the location data and/or to communicate them to the device mentioned above and connected to the electronic control system for communication.

The electronic control system can have a communications interface for communicating data to another device. The other device can, for example, be the previously mentioned operating device or a server, which is configured to store the data, e.g., in a central and/or decentralized database, and to provide them to software, e.g., a web or cloud application, for display or for data analysis. The software can be run on the operating device, the server, or on a further device, e.g., a further server or a PC, or in a cloud, of which the server may be a part. This enables the liquid sampling and/or sampling station-specific data, such as, for example, identifiers, time stamps, temperature data, weather data, location data (e.g., GPS data of the sampling station), or measured values of the sample liquid, to be linked to findings, which are determined in the laboratory and can also be imported into the operating device or the software, of the qualitative and/or quantitative determination of the at least one analyte, hereafter also referred to as analytical findings for short, in the samples provided by the sampling station. In addition, the data, measured values, and analytical findings thus determined of samples taken at different sampling stations can, in a web or cloud application, be combined and compared with one another and/or with data from other sources.

The invention also comprises a measuring system for the qualitative or quantitative determination of at least one analyte, in particular a biopolymer, in a sample liquid, in particular water or wastewater, comprising: - an automatic sampling apparatus for taking liquid samples of the sample liquid at a sampling station, e.g., a body of water or container, in particular a pipe in a water supply or wastewater network, or an inflow or a basin of a sewage treatment plant, according to one of the embodiments described above;

- an analytical instrument configured for the qualitative or quantitative determination of the at least one analyte; and - a transfer device configured to provide a sample of the at least one analyte that is concentrated or extracted by means of the sampling apparatus.

If the sampling apparatus comprises, for example, a collection matrix having functionalized magnetic beads to which the at least one analyte selectively binds, the transfer device can comprise a magnet which is configured to attract the magnetic beads after contact with the sample liquid and to transfer them into a further vessel. In this way, the collection matrix with the analyte bound thereto can be separated from the other constituents of the liquid sample. If the collection matrix is contained, as mentioned further above, in an insert of the sample container or in a removable cartridge, the transfer device can be configured to automatically remove the cartridge or the insert. The collection matrix with the bound analyte can be provided, for example, as a sample of the analyte, which is concentrated or extracted by means of the sampling apparatus for further analysis by means of the analytical instrument.

Furthermore, the transfer device can comprise means for eluting the analyte from the collection matrix - here, for example, from the magnetic beads. In this case, the eluate can be provided as a sample of the analyte that is concentrated or extracted by means of the sampling apparatus. In an advantageous embodiment, the transfer device is further configured to transfer the provided sample to the analytical instrument.

The measuring system can further comprise an electronic control system configured to control the transfer device for the automatic transfer of a sample of the concentrated or extracted analyte to the analytical instrument. For example, the electronic control system can control pumps, valves, and movable parts, such as, for example, the aforementioned magnet, in order to move the collection matrix out of an original position in the sample vessel or in a flow-through unit in the fluid flow path of the sampling apparatus into the analytical instrument or into a further container belonging to the transfer device, and to elute the analyte from the collection matrix. The electronic control system can be the electronic control system of the automatic sampling apparatus mentioned further above or can be part of the electronic control system of the automatic sampling apparatus.

The analytical instrument can be designed to carry out the qualitative or quantitative determination of the analyte partially or completely automatically. The analysis by means of the analytical instrument can take place, for example, on the basis of an affinity-based, in particular antibody-based, immunological assay or on the basis of a real-time, qPCR-based method. In a further possible embodiment, the analytical instrument can be configured to carry out the qualitative or quantitative determination of the analyte by means of a spectroscopic method, e.g., by means of Raman spectroscopy, and in particular also by means of surface-enhanced Raman spectroscopy (SERS) or tip-enhanced Raman spectroscopy (TERS), by means of mass spectrometry, or by means of hyperspectral imaging.

The analytical instrument can have a computing unit which is configured to determine a qualitative or quantitative analytical finding, e.g., an analyte concentration in the original liquid sample, and which is further configured to control the analytical instrument for the automatic determination of the analyte. The computing unit can be configured to store analytical findings of the qualitative and/or quantitative determinations in the samples provided by the transfer device, in particular so as to be linked in each case to an identifier of the liquid sample from which a sample provided by the transfer device was obtained. The computing unit of the analytical instrument can further have a communications interface for the communication of data to another device. The other device can, for example, be the electronic control system of the sampling apparatus, the operating device already mentioned further above, or the previously mentioned server which is configured to store data made available by the analytical instrument, e.g., in a central and/or decentralized database, and to provide them to software, e.g., a web or cloud application, for display or for data analysis. The software can be run on the operating device, the server, or on a further device, e.g., a further server or a PC, or in a cloud, of which the server may be a part. As already mentioned, the software can link the analytical findings provided by the analytical instrument to data of the associated liquid samples acquired by the sampling apparatus and perform further data analysis with the associated data.

The invention also comprises a method for the automatic provision of a sample for a qualitative and/or quantitative determination of at least one analyte, in particular a biopolymer, in a sample liquid, in particular water or wastewater. The method comprises the following steps: - taking a definable volume of the sample liquid, in the form of a liquid sample, from the sampling station by means of an automatic sampling apparatus, in particular the sampling apparatus according to one of the embodiments described above, from the sample liquid present at a sampling station, e.g., in a container or a body of water, in particular in a pipe in a water supply or wastewater network, or at a discharge point to a wastewater network, or in an inflow or a basin of a sewage treatment plant; - transporting the volume of the sample liquid, in the form of a liquid sample, into a sample container; and - bringing the liquid sample into contact with a collection matrix that immobilizes, and in particular substantially selectively binds, the analyte.

In an alternative embodiment, the method can also, instead of bringing the liquid sample into contact with the collection matrix, include the extraction or concentration of the analyte by means of centrifugation or by means of a precipitation reaction.

As already described above in connection with the sampling apparatus, the collecting matrix can be placed in the sample container or be arranged in a flow-through unit fluidically connected to the sample container, in particular a fluid line or a flow-through filter cartridge. The collection matrix can be designed as previously explained in connection with the sampling apparatus.

The method may further comprise removing the collection matrix with the analyte from the device and/or eluting the analyte from the collection matrix to form a solution of the analyte as a sample for subsequent qualitative or quantitative determination. The subsequent qualitative or quantitative determination of the analyte can be carried out by means of an affinity-based, in particular antibody-based, immunological assay by means of a real-time, qPCR-based method or by means of a spectroscopic method. The findings of this determination are referred to above and below as the analytical findings. The analytical findings can represent the concentration of the analyte in the provided sample and/or the concentration of the analyte in the original liquid sample.

Furthermore, the method can comprise storing an identifier of the liquid sample and a time stamp, representing the time the liquid sample was taken from the sampling station, in an electronic control system of the automatic sampling apparatus. The identifier of the liquid sample and the associated time stamp can be stored by the electronic control system and/or by a device that can be connected to the electronic control system for communication, e.g., an, in particular portable, operating device or a server, and can be made available to software, e.g., a web or cloud application, which links the stored data to further data and optionally performs further data analyses.

In addition, the method can comprise acquiring at least one measured value of at least one measurand of the sample liquid, e.g., the temperature, the pH, or the conductivity at the time the liquid sample was taken from the sampling station, by means of an additional sensor, and storing the measured value in the electronic control system for identifying the liquid sample. Further measurands, which can be measured by means of one or more additional sensors that can be connected to the electronic control system, are turbidity, concentrations of dissolved gases, ion concentrations, or concentrations of photometrically or spectrometrically detectable molecules or cumulative parameters, such as SAC value (spectral absorption coefficient), COD (chemical oxygen demand), or TOC (total organically-bound carbon). The at least one additionally determined measured value, in particular several additionally determined measured values of one or more different parameters of said parameters, can be stored so as to be linked to the identifier of the liquid sample. They can be taken into account when determining or evaluating the analytical findings determined using the provided sample. For example, the at least one additional measured value can be included in a calculation model for determining the concentration of the analyte in the original liquid sample from a qPCR measurement result or from a measured value of an immunological assay or from spectrometric measurement data.

A pH value measured in the sample liquid can also be used to set, by means of the electronic control system, a pH value in the liquid sample that is advantageous for binding to the collection matrix. For this purpose, the sampling apparatus can have storage containers with reagents which influence the pH value of the liquid sample, fluid lines, one or more pumps, and, optionally, one or more valves which are actuated by the electronic control system for setting the pH of the liquid sample.

The method can further comprise acquiring a temperature or a temperature profile to which the liquid sample is exposed from the time of its removal. The temperature or the temperature profile can be stored by the electronic control system and linked to the stored identifier of the liquid sample. The temperature profile to which the liquid sample was exposed can be included in an evaluation and/or determination of the analytical findings. For example, the stored temperature or the stored temperature profile can be included in a calculation model for determining the concentration of the analyte in the original liquid sample from a qPCR measurement result, from a measured value of an immunological assay, or from a spectroscopic measurement.

The method can further comprise acquiring location data representing the location of the sampling station, e.g., by manual input or by a GPS receiver, and storing the location data in the electronic control system.

The method can further comprise the transfer of the identifier of the liquid sample and further data, which are stored so as to be linked with the identifier in the electronic control system, e.g., a time stamp associated with the identifier, measured values associated with the identifier, location information, and/or sampling station-specific data associated with the identifier, to an operating device or to an analytical instrument used for the analysis of the provided sample, in particular to a laboratory instrument, or to a server, which is connected to the analytical instrument and/or to the electronic control system for wireless or wired communication and which stores the data obtained from the electronic control system and/or the analytical instrument, the memory of which server can be accessed by software, e.g., a web or cloud application, in order to display and/or perform a further analysis of the data. The software can be run on the operating device, the server, or another device.

The invention also comprises a method for the qualitative or quantitative determination of at least one analyte, in particular a biopolymer in a sample liquid, in particular water or wastewater, comprising: - providing a sample by means of the method described above in one of its specific method configurations, likewise described above, and - qualitative or quantitative determination of the analyte by means of an analytical method, in particular by means of an affinity-based, in particular antibody-based, assay, by means of a real-time, qPCR-based method or by means of a spectroscopic method.

The method can be carried out with the measuring system described further above.

The analytical method can be carried out completely or partially automatically on the provided sample by means of at least one analytical instrument, which comprises a computing unit configured to determine qualitative or quantitative analytical findings - for example, an analyte concentration in the original liquid sample.

The analytical instrument can be an online analytical instrument that is located directly at the sampling station. Alternatively, the sample provided can be taken to a laboratory and analyzed there partially or completely automatically by means of at least one laboratory analytical instrument. The sample provided according to the method described above, which is obtained by concentration of the analyte by means of a collection matrix or, alternatively, also according to another method, e.g., centrifugation, is less complex to transport than a sample container containing the entire liquid sample. Due to the correspondingly smaller volume, cooling during transport is also easier to accomplish.

As already described above, the method can comprise determining a concentration of the analyte in the original liquid sample, with additional inclusion of at least one measured value acquired at the sampling station, e.g., the temperature, the pH value, or the conductivity, or one or more further measurands, wherein the measured value is provided by the electronic control system of the sampling apparatus. The concentration of the analyte in the original liquid sample also corresponds to the concentration of the analyte in the sample liquid that was present at the sampling station at the time of sampling.

The time of sampling can be represented by a time stamp which is recorded by the electronic control system of the sampling apparatus and is stored in the electronic control system so as to be linked to the identifier of the liquid sample, and/or can be output by the electronic control system to another device, e.g., the analytical instrument, to an operating device, or a server, for storage linked to the identifier of the liquid sample. The sampling station can be represented by location information, e.g., GPS data, which can be acquired and stored by the electronic control system and/or can be output so as to be linked with the identifier of the liquid sample to another device, e.g., the analytical instrument, to the operating device, and/or to a server, for storage linked with the identifier of the liquid sample.

The electronic control system of the sampling apparatus can transmit the measured values, to be included in the determination of the concentration of the analyte in the liquid sample, together with the identifier of the liquid sample, to the analytical instrument for storage. Alternatively, the values, together with the identifier of the liquid sample from the electronic control system, can be imported into an operating device for storage and output from the operating device to the analytical instrument and stored there. In a further variant, the values, together with the identifier of the liquid sample, can be output from the electronic control system to a server which stores the values and provides them to software - for example, a web or cloud application. The analytical instrument or the processing unit of the analytical instrument can output the analytical findings of the sample, e.g., a concentration of the analyte determined in the liquid sample, to the electronic control system of the sampling apparatus or to the operating device or the server or another server, wherein the electronic control system, the operating device, the server, or the other server stores the analytical findings so as to be linked to the identifier of the liquid sample. The software can access the operating device, the server, or the other server in order to read out the stored analytical findings and the associated identifier, and to use them for further data analysis.

The quantitative determination of the analyte, e.g., the determination of an analyte concentration in the original liquid sample, taking into account the at least one further measured value, can be carried out in one embodiment of the method by the processing unit of the analytical instrument using the data provided by means of the operating device or the software. In a further embodiment of the method, the quantitative determination of the analyte by the software can be carried out using analytical findings determined from the sample by the processing unit of the analytical instrument and output to the server, and, optionally, using measured values acquired by the electronic control system of the sampling apparatus and output to the server.

The calculation of the analyte concentration in the original liquid sample, taking into account one or more measured values, can be carried out on the basis of a calculation model. The calculation model can take into account, for example, the influence of the temperature on the binding behavior of the analyte to a collection matrix for concentration and/or extraction of the analyte in the liquid sample, and the influence of the temperature on the degradation process. In this model, measured temperature values acquired by the sampling apparatus or measured values of the temperature profile, to which the liquid sample was exposed before analysis, can be included.

The software can be run on the operating device, the server, another server, or distributed in a cloud.

The additional measured values acquired by the electronic control system and associated with the liquid sample can not only be used to determine a concentration of the analyte, but can, alternatively or additionally, be used to evaluate the analytical findings or to create or adapt chemometric or epidemiological models. They can also be used for statistical purposes.

The method can further comprise communicating the analytical findings and, optionally, additional data from the electronic control system and/or the analytical instrument to the mentioned software, e.g., a web or cloud application, for further analysis. In addition to the additional measured values already mentioned and the time stamp assigned to the liquid sample, the additional data can be an identifier of the sampling station, e.g., GPS data or other location data, which identify the sampling station.

The software, in particular in the form of a cloud application, can access data of a plurality of sampling apparatuses at a plurality of sampling stations. For example, the data, in particular identifiers of liquid samples, can be stored, linked with associated time stamps, location information, and measured values, in a central database or a database in a cloud distributed over several computers, which the software can access. The analytical findings, which are obtained by means of the qualitative or quantitative determinations of the analyte carried out by means of the liquid samples collected by the sampling apparatuses, can also be stored in the database so as to be linked to the identifiers of the associated liquid samples. The software can use the data for comprehensive data analysis. For example, it can monitor a time profile of the analytical findings and thus provide an early warning system, which detects an increase in the concentration of the analyte at a sampling station or in a region comprising several sampling stations, and generates a warning message on the basis thereof.

The software can also use the data for analysis of a temporal and/or spatial development of the analyte concentration in an extended area, e.g., in a water or wastewater network of a larger region, or an extensive body of water. It can also provide a visualization of the analyte concentration in the form of a map. Trends, i.e., temporal developments, can also be represented on the map.

The software can provide the findings of the data analysis, in particular the aforementioned displays of information of an early warning system or graphical representations of maps, to other computers (terminals), in particular in a browser-based application on the terminals.

The above devices and methods are suitable, for example, for monitoring water or wastewater networks, regions in sewage plants, and discharge points in wastewater networks for the occurrence of, or for trends in, the concentration of pathogens, e.g., viruses or bacteria, or parts of pathogens, e.g., DNA, RNA, or shell and envelope components, in the water. This can be used for modeling, predicting, and analyzing epidemics, in particular as an early warning system. Such a pathogen can, for example, be the Sars-CoV-2 virus.

The invention is explained in further detail below on the basis of the exemplary embodiments shown in the figures. The following are shown:

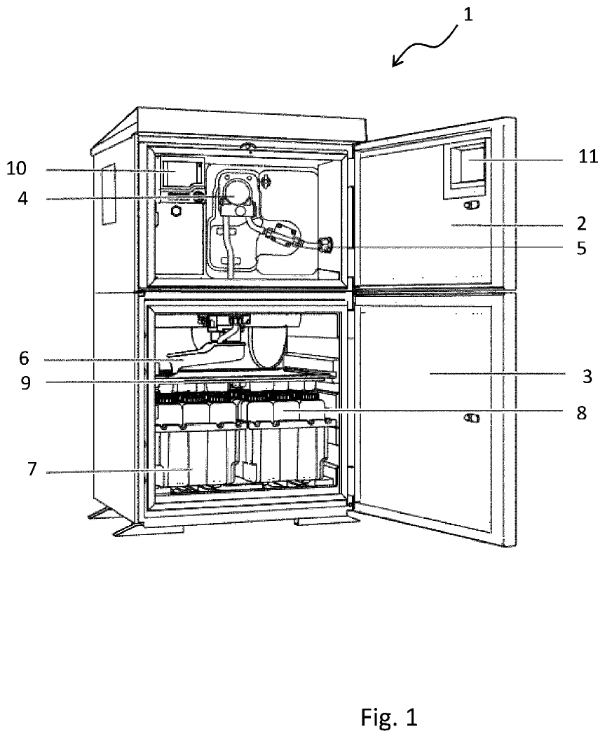

FIG. 1 an automatic sampling apparatus;

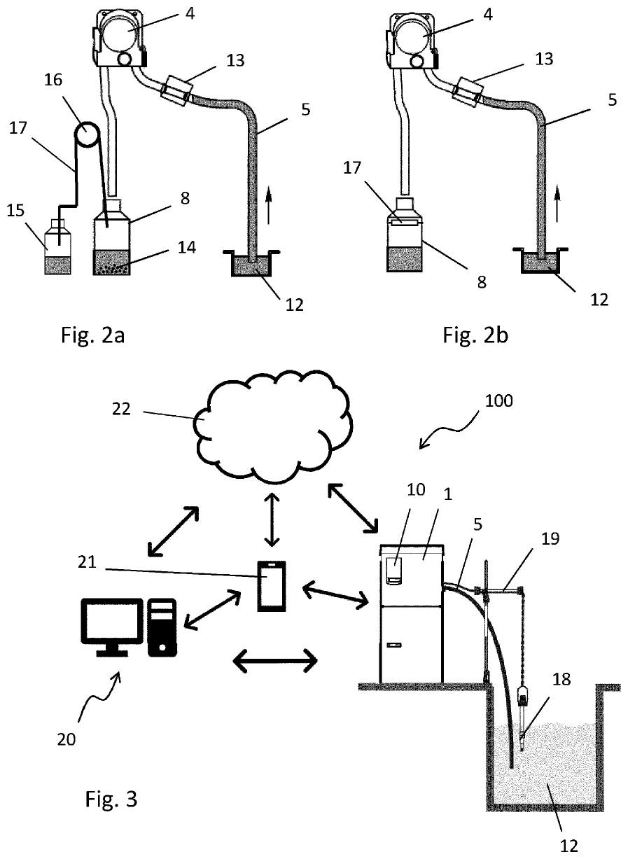

FIG. 2a a detail of the automatic sampling apparatus in a first embodiment;

FIG. 2b a detail of the automatic sampling apparatus in a second embodiment; FIG. 3 a measurement system for the qualitative or quantitative determination of an analyte in a wastewater sample in a first embodiment; and

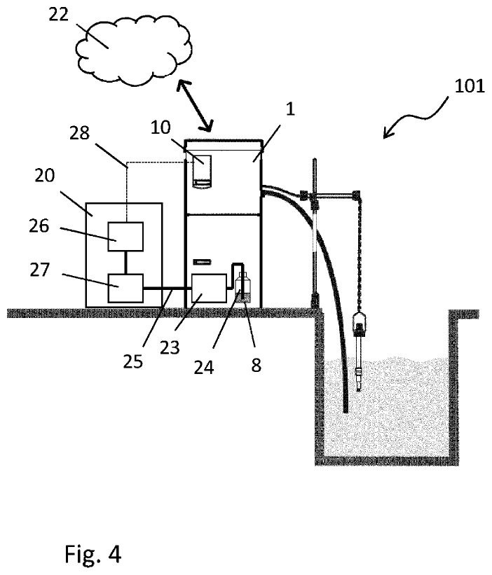

FIG. 4 a measuring system for the qualitative or quantitative determination of an analyte in a wastewater sample in a second embodiment.

FIG. 1 depicts an automatic sampling apparatus 1. It is designed as a cabinet device which is divided into an upper metering chamber and a lower sample chamber. The metering chamber can be closed with a metering chamber door 2, and the sample chamber with a sample chamber door 3. The sample chamber is, optionally, cooled. A pump 4 is arranged in the metering chamber and is designed as a hose pump in the present example. Other embodiments, e.g., as a vacuum pump, are possible. The pump 4 interacts with a sample line 5, which in the present example is designed as a hose line.

The sample line 5 can be fluidically connected at its first end (not visible in FIG. 1) to a sampling station 12 (FIGS. 2a, 2b) from which liquid samples of a sample liquid are to be taken for the analysis. The sampling station can be an open body of water or channel. In this case, the fluidic connection can be established by immersing the first end of the sample line 5. The sampling station 12 can also be a closed container, e.g., a pipeline, through which the sample liquid flows. In this case, the fluidic connection can be produced by means of a sampling valve.

The second end of the sample line 5 is fluidically connected to a sample distributor 6, embodied here as a rotary arm. Located in the metering chamber are two bottle baskets 7 in which several sample containers 8 - in this case in the form of plastic or glass bottles - are accommodated.

The sample distributor can be moved away over the sample containers 8 by means of a distributor mechanism—here, a rotary mechanism - in order to fluidically connect the sample line 5 to one of the sample containers 8 in each case. If the tip of the rotary arm is located above the opening of a sample container 8, a fluid flow path is formed which runs from the sampling station 12 via the sample line 5 and the sample distributor 6 into the sample container 8. In the present example, a distributor plate 9 is arranged above the sample container 8 for directing the liquid along the fluid flow path. However, this is only optionally present.

At the first end of the sample line 5, a filter or a filter cascade can be arranged which, by means of size filtration, retains at least a portion of solids contained in the sample liquid. The filter or filters can also be arranged at another location of the fluid flow path between the first end of the sample line 5 and the sample container 8.

The sampling apparatus 1 further comprises an electronic control system 10, which is configured to control the pump 4 and the sample distributor 6 for transporting a definable volume of the sample liquid from the sampling station into a sample container 8. A window 11 is arranged in the metering chamber door 2, through which a display of the electronic control system 10 can be seen and the electronic control system 10 can be operated by means of input buttons and a rotary/push switch, even when the metering chamber door is closed.

The electronic control system 10 has a processor and memory in which operating and evaluation programs are stored, which the electronic control system 10 can implement to control the sampling apparatus 1 for the sampling and for acquiring and storing data in connection with the sampling. The sampling apparatus 1 can optionally have a cooling device which serves to cool the sample chamber. A temperature of the sample chamber can be adjustable by means of the electronic control system 10.

The electronic control system 10 can, in particular, be configured to store an identifier, as well as a time stamp associated with the identifier, to a sample transported into a sample container 8, said time stamp identifying the time of the sampling. The electronic control system 10 can assign and save further information to the identifier, e.g., measurement data of further sensors connected to the electronic control system 10, such as temperature measurement data, pH measurement data, conductivity measurement data, turbidity measurement data, measured value profiles of said parameters, and location data, e.g., the location of the sampling station at which the sampling apparatus 1 is arranged.

The electronic control system 10 also has one or more communications interfaces via which it can communicate with further data processing devices, e.g., with an analytical instrument which serves to analyze the samples provided by the sampling device 1, or with a portable operating device by means of which an operator can read data from the electronic control system 10 or output them to the electronic control system 10. The electronic control system 10 can also be configured to transmit data to a server which stores the data and makes them available to software - for example, a web or cloud application. These data can comprise the identifiers and associated data of collected liquid samples, as well as location data characterizing the sampling station. The software can access these data for further evaluation and analysis. Conversely, the software can provide data or information for future sampling or for display on a user interface to the electronic control system 10 via the server of the electronic control system 10.

The electronic control system 10 can have one or more of the following types of communications interfaces: communications interfaces according to a standard of the process industry, e.g., 4 . . . 20 mA, Profibus, HART, Modbus, or other, in particular, also proprietary, communications standards, but also communications interfaces for communication according to an Ethernet standard or a Bluetooth standard.

The sampling apparatus 1 also has means for concentration and/or extraction of an analyte that is taken from the sampling station and comprises a biopolymer. The analyte may, for example, be a virus or virus component, or a bacterium or a bacterial component. Components of viruses and bacteria are understood here to mean, in particular, parts of their shells or envelopes, in particular proteins, as well as polynucleotides, such as RNA or RNA fragments, or DNA or DNA fragments. Examples of agents for concentration and/or extraction of such an analyte are described below with reference to FIGS. 2a and 2b.

FIGS. 2a and 2b schematically show the fluid flow path between the sampling station 12 (here, an indicated channel) and a sample container 8 (the sample distributor has been omitted in this illustration for the sake of clarity). Located in the fluid flow path is a flow meter 13, which serves to adjust the definable sample volume. The definable volume of the sample liquid can be transported via the sample line 5 into the sample container 8 by means of the pump 4.

In the example shown in FIG. 2a, a collection matrix in the form of a plurality of magnetic or polymer beads 14 is provided in the sample container 8, the surfaces of which magnetic or polymer beads are modified in such a way that the analyte in the sample liquid binds substantially selectively to the surfaces. In the main, if the analyte is, for example, virus RNA of a specific virus species, RNA of such viruses binds to the beads, but not—or at least to a very much lesser degree - other RNA or DNA located in the sample liquid. Such beads are known in the prior art for the enrichment and/or extraction of biomolecules, in particular biopolymers. Their surfaces can be modified by structuring or functionalizing with antibodies or other capture structures, in order to provide the desired selectivity.

The selective binding of the analyte to the collection matrix can be optimized by adjusting an appropriate pH value in the liquid sample. To adjust the pH, the sampling apparatus can comprise one or more further liquid containers 15 having one or more reagents, e.g., an acid, a base, and/or a buffer solution, and a further pump 16, by means of which the reagent or the reagents can be added to the liquid sample. In the present example, the further pump 16 is a hose pump which interacts with a fluid line 17 in order to transport a reagent from the liquid container 15 into the sample container 8. The pump 16 can be controlled by the electronic control system 10 of the sampling apparatus 1 to meter a quantity of the reagent or the reagents required to achieve a desired pH value. For this purpose, a pH sensor can be provided in the sample container, which sensor outputs measured pH values to the electronic control system 10 so that this can control the addition of the reagent or the reagents on the basis of the measured pH values. Alternatively, the electronic control system 10 can also be connected to a pH sensor immersed at the sampling station 12 in the sample liquid present there and use measured pH values of said sensor to control the addition of the reagent or the reagents to the liquid sample in the sample container 8.

The beads 14, with the analyte bound thereon or immobilized, can be removed manually or by means of a (partially) automatic transfer device (not shown in FIG. 2a) from the sample container 8 and transported into a laboratory for further analysis. This has the advantage that only the beads 14 have to be transported and optionally cooled during transport, and not the large-volume sample containers 8. In order to remove magnetic beads, a magnet, in particular a switchable electromagnet, can be used, which can be moved, for example, in an automated manner for removing the magnetic beats and can be controlled by the electronic control system 10. If the beads 14 are not magnetic, another possibility for removing the beads 14 from the sample container 8 can consist in automatically emptying the sample container 8 and, in so doing, retaining the beads 14 in a filter. The beads 14 can also be held in an insert in the sample container 8 and removed from the sample container 8 by removing the insert. If a transfer device is present, it can also comprise means for eluting the analyte from the beads, so that the sample provided for transport to the laboratory or for transfer to an analytical instrument for further qualitative or quantitative analysis is formed from the eluate.

FIG. 2b shows another embodiment of the means for concentration and/or extraction of the analyte. Here, these means comprise a removable cartridge 17 arranged in the upper region of the sample container 8, which cartridge contains an analyte-affine collection matrix. This can, for example, be a functionalized nonwoven, a chromatography column, or a packed bed of surface-functionalized polymer beads. The beads or the nonwoven can be functionalized in a manner analogous to the magnetic or polymer beads used in the example of FIG. 2a. While, during sampling, the sample liquid flows through the cartridge 17 on the path into the sample container 8, the analyte in the cartridge is bound substantially selectively to the collection matrix or, in the case of a chromatography column, is separated chromatographically from further sample constituents and retained in the collection matrix.

The cartridge 17 can also be arranged outside the sample container 8 at a different position in the fluid flow path. The cartridge 17 can be removed automatically or manually from the sample container or from the fluid flow path in the sampling apparatus 1 and transported to a laboratory for further analysis. If the cartridge 17 is removed automatically by means of a transfer device, the latter can additionally comprise means for eluting the analyte from the beads, as in the above-described example, so that the sample provided for transport into the laboratory or for transfer to an analytical instrument for the further qualitative or quantitative analysis is formed from the eluate. Further examples of means for concentration and/or extraction of the analyte are conceivable —for example, a centrifugal device which can be integrated into the sampling apparatus 1.

FIG. 3 schematically illustrates a measuring system 100 for the qualitative or quantitative determination of the analyte contained in the sample liquid by means of liquid samples taken from the sampling station 12.

The measuring system 100 comprises the sampling apparatus 1, previously described with reference to FIGS. 1, 2a, 2b, with the electronic control system 10. The sample line 10 of the sampling apparatus 1 is fluidically connected to the sampling station 12, which, in the present example, is an open basin—for example, a basin or an inflow of a sewage treatment plant. An additional sensor 18 is immersed in the sample liquid present at the sampling station 12, which additional sensor 18 is fastened to a valve 19 and is connected via a cable to the electronic control system 10 of the sampling apparatus 1 for bidirectional communication. The additional sensor 18 can have a conductivity sensor, a pH sensor, a turbidity sensor, a temperature sensor, a photometric or spectrometric probe for determining a concentration of one or more substances, in particular a nitrate or SAC probe, or an ion-selective electrode. The additional sensor 18 outputs measured values to the electronic control system 10 of the sampling apparatus 1. As already mentioned in connection with FIG. 1, the electronic control system 10 is configured to link an identifier of a taken liquid sample, as well as a time stamp characterizing the time of sampling, and further data, in particular measurement data of the additional sensor 18, with the identifier and store it. Of course, the sampling apparatus 1 can also have several additional sensors 18 connected to the electronic control system 10, the measured values of which can be acquired and stored.

The measuring system 100 also has an analytical instrument 20 arranged in a laboratory remote from the sampling station 12, which analytical instrument comprises a liquid handling system for sample preparation and processing, as well as an analysis unit for subsequent automatic performance of an immunological assay or real-time, qPCR-based, or spectroscopic analysis of the sample, as well as a processing unit. The sample obtained from a liquid sample taken from the sampling apparatus 1 by concentration and/or extraction of the analyte for further analysis is transferred to the analytical instrument 20 and analyzed automatically or partially automatically from the sample by means of these methods known per se in the prior art. The processing unit serves, among other things, to calculate quantitative analytical findings, e.g., a concentration of the analyte in the sample made available by the sampling apparatus 1 or in the original liquid sample, and, optionally, also to control liquid handling and analytical procedures carried out automatically by means of the analytical instrument 20.

As indicated in FIG. 3 by a double arrow, the analytical instrument 20 can be connected to the sampling apparatus 1 for communication—for example, via a LAN connection (intranet or internet). Via this connection, the sampling apparatus 1 can transmit the identifiers of liquid samples and associated information, such as sensor data, time stamps, and location information, to the processing unit of the analytical instrument 20. The processing unit of the analytical instrument 20 can, conversely, transmit analytical findings, together with the identifier of the associated liquid samples, to the sampling apparatus 1. The processing unit of the analytical instrument 20 can use the additional information obtained from the sampling apparatus 1 to determine and/or evaluate the analytical findings of the individual samples. For example, when determining quantitative analytical findings, the influence of further measurands of the sample liquid, e.g., the pH value or the conductivity of the sample liquid at the time of sampling, can be taken into account. The analytical findings determined by the analytical instrument 20 and the additional information provided by the sampling apparatus 1 are combined on the basis of the identifiers of the samples.

The measuring system 100 can optionally comprise a mobile operating device 21, which, in the present example, is designed as a smartphone or tablet. In the present example, the operating device 21 can be configured for bidirectional communication both with the sampling apparatus 1 and with the analytical instrument 20. It can therefore be used to transmit data from one unit to the other. It can also be used for importing an identifier which is visibly mounted on the sample container 8 or mounted readably in an NFC or RFID chip on the sample container 8, in order to avoid confusion when the concentrated samples are transferred from the sampling apparatus 1 to the analytical instrument 20 in the laboratory. The methods and means used for the clear identification of sample containers for identifying and managing collected samples in sample containers during transport between a sampling station and a laboratory are sufficiently known to the person skilled in the art and can also be used here. Therefore, no further details are given here.

In the present example, the operating device 21, the analytical instrument 20, and the electronic control system 10 of the sampling apparatus 1 are designed for communication with a server, which, in the present example, is part of a cloud 22. The server can also be a local computer, i.e., a computer located at the sampling station or in the laboratory, or a single central computer, which can communicate with the processing unit of the analytical instrument 20, the operating device 21, and/or the electronic control system 10 via intranet or internet.

The server can collect, store, and continue to evaluate data, measured values, and analytical findings linked with the identifier of the associated sample. The storage can take place in a central database or a database distributed in the cloud. Software can be run on the server, on a computer that can be connected to the server, e.g., another server, a PC, or the operating device, or in the cloud 22, which software accesses the stored data and performs further data analyses and/or can display data and results of the data analyses. This software can be a web or cloud application, for example.

The software or cloud 22 can be connected to a plurality of sampling stations and analytical laboratory instruments, and thus determine and analyze a plurality of data from spatially-—distributed sampling stations. Such a cloud application can detect and evaluate quantitative or qualitative analytical findings for certain analytes, such as viruses or other microorganisms, in particular pathogens, in a body of water, a water or wastewater network, or in a multiplicity of different private and/or municipal sewage plants. It can monitor temporal developments of the concentration of the analyte, e.g., a virus concentration at the individual sampling stations or in a larger region comprising several sampling stations, and thus serve as an early-warning system for the outbreak of a disease or epidemic in the monitored region. If the cloud application has data from samples collected and analyzed at a plurality of sampling stations, it can provide a map of the concentrations of the analyte at the various sampling stations.

The cloud application 22 can be configured to collate the analytical findings and other data determined for the individual sampling stations with data from further sources. For example, if the analyte is a virus or a component of the virus, e.g., virus RNA, and if the sampling stations considered are channels or containers in wastewater networks or sewage treatment plants, then the analytical findings thus obtained that reflect the concentration of the virus or the virus RNA in the regional wastewater can be collated with data from the regional health authorities in order to predict breakouts of a disease caused by the virus at an early stage, to increase the number of tests performed locally on patients, or to identify discrepancies between tests on patients and analytical findings in the wastewater.

FIG. 4 shows a further exemplary embodiment of a measuring system 101 which serves for the qualitative or quantitative determination of the analyte contained in the sample liquid by means of liquid samples taken from the sampling station 12. Like the measuring system 100, which has already been described with reference to FIG. 3, the measuring system 101 also comprises the sampling apparatus 1 described above with the additional sensor 18 and the electronic control system 10, which is configured for controlling the sampling and for acquiring and storing identifiers of liquid samples, and thus linked additional data, e.g., time and location data and additional measured values.

Furthermore, the measuring system 101 comprises an analytical instrument 20 which serves for the qualitative or quantitative determination of the analyte in samples made available by the sampling apparatus 1.

Unlike in the example of FIG. 3, the measuring system 101 described here has a completely automatic transfer device 23 configured to automatically extract a collection matrix, which is contained in a sample container 8 of the sampling apparatus 1 and substantially selectively binds the analyte, from the sample container 8. In the present example, the collection matrix is formed from a plurality of surface-modified magnetic beads, which are placed in the sample container 8 such that analyte present in the liquid sample forms on the magnetic beads. As described further above, means for adjusting the pH value in the liquid sample can additionally be provided. To remove the collection matrix from the sample container 8, the transfer device 23 has one or more movable and/or switchable magnets 24 with which the magnetic beads can be attracted and removed from the sample container 8. The magnetic beads are rinsed with solvent in the transfer device 23, and the analyte is eluted from the collection matrix. The liquid sample thus obtained is transferred from the transfer device 23 via a fluid line 25 into the analytical instrument 20. The transfer unit can be controlled by means of a local control unit or by means of the electronic control system 10 of the sampling apparatus 1.

The analytical instrument 20 can be designed analogously to the analytical instrument described above with reference to FIG. 3 for the automatic execution of a further sample preparation and for subsequent performance of an analysis, e.g., an immunological assay, of a real-time, qPCR-based analysis or a spectroscopic analysis of the sample. The analytical instrument 20 comprises a computing unit 26 and an analytical cartridge 27 in which the liquid analysis is controlled by means of the processing unit 26. The processing unit 26 is connected to the control unit 10, for communication via cables 28 or wirelessly, in order to import identifiers and associated data of the samples to be analyzed and/or return analytical findings to the electronic control system 10.

The electronic control system 10 and/or the computing unit 26 can communicate with a server, which, in the present example, is part of a cloud 22. Data exchange and analysis, as well as further evaluations of the provided data and analytical findings by means of a cloud application, which accesses the data stored in the server, can take place in a very similar manner to that described above with reference to FIG. 3

The sampling apparatus 1 described here and the measuring systems 100 and 101 can serve for the process-capable, systematic taking of liquid samples, in particular from water supply networks, wastewater networks, and sewage plants, with subsequent analysis for the qualitative or quantitative determination of analytes comprising biopolymers, such as viruses, bacteria, or DNA or RNA. The data can be provided systematically, in regular measuring cycles and with little outlay in terms of personnel and time, and, advantageously, by a web or cloud application which a plurality of terminals can access, and which enables the linking of the sample data to further data from other sources, to be analyzed further or made available for further analyses by authorities or scientific research facilities. Therefore, on the one hand, comprehensive studies of the course of epidemics are possible, and, on the other, a reliable early warning system can be made available in this way when pathogens occur in the water supply or wastewater.

1-26. (canceled)

27. An automatic sampling apparatus for taking liquid samples of a sample liquid present at a sampling station of a body of water or of a container for the qualitative or quantitative determination of at least one analyte contained in the sample liquid, the apparatus comprising:
a sample line that can be fluidically connected to the sampling station;
a pump device;
at least one sample container that can be fluidically connected to the sample line; and
an electronic control system which is configured to fluidically connect the sample line to the at least one sample container such that a fluid flow path extending from the sampling station through the sample line into the sample container is formed,
and wherein the electronic control system is further configured to transport, using the pump device, a definable volume of the sample liquid, in the form of a liquid sample, along the fluid flow path into the sample container,
wherein the sampling apparatus concentrates or extracts the analyte present in the liquid sample.

28. The automatic sampling apparatus of claim 27, wherein the concentration or extraction includes a collection matrix which can be brought into contact with the liquid sample and which substantially selectively binds the analyte.

29. The automatic sampling apparatus of claim 28, wherein the collection matrix is arranged in the at least one sample container or in a flow-through unit fluidically connected to the sample container.

30. The automatic sampling apparatus of claim 28, wherein the collection matrix has a plurality of particles, a surface-functionalized substrate having a roughened or structured or porous surface, a functionalized nonwoven, or a chromatographic separation column.

31. The automatic sampling apparatus of claim 30, wherein the particles or the surface are functionalized for selective interaction with the analyte.

32. The automatic sampling apparatus of claim 27, wherein the concentration or extraction includes using a centrifuge integrated into the apparatus.

33. The automatic sampling apparatus of claim 27, wherein the electronic control system is configured to store an identifier of the liquid sample for each liquid sample taken from the sampling station, and to save a time stamp, indicating the time at which the liquid sample is taken from the sampling station, to the identifier.

34. The automatic sampling apparatus of claim 33, further comprising at least one interface for connecting the electronic control system to at least one sensor which can be brought into contact with the sample liquid present at the sampling station, and which is designed to generate measurement signals of at least one measurand of the sample liquid,
wherein the electronic control system is further configured to save at least one measured value, derived from the measurement signals, of the measurand, which is acquired at the time at which the liquid sample is taken from the sampling station, to the identifier of the liquid sample.

35. The automatic sampling apparatus of claim 33, further comprising a temperature sensor which is designed to acquire measured values representing a temperature of a liquid sample contained in the at least one sample container and to output these values to the electronic control system, and
wherein the electronic control system is further configured to save at least one of the measured values or a measured value profile to the identifier of the liquid sample.

36. The automatic sampling apparatus of claim 33, further comprising a GPS receiver which is configured to output location data to the electronic control system, wherein the electronic control system is configured to save the location data to the identifier of the liquid sample.

37. The automatic sampling apparatus of claim 27, wherein the electronic control system has a communications interface for outputting data to an operating device or a server, wherein the server is configured to store the data and to provide it to a non-transitory computer readable medium.

38. A measurement system for the qualitative or quantitative determination of at least one analyte in a sample liquid, comprising:
an automatic sampling apparatus for removing liquid samples of the sample liquid at a sampling station, wherein the automatic sampling apparatus includes:
a sample line that can be fluidically connected to the sampling station;
a pump device;
at least one sample container that can be fluidically connected to the sample line; and
an electronic control system which is configured to fluidically connect the sample line to the at least one sample container such that a fluid flow path extending from the sampling station through the sample line into the sample container is formed,
wherein the electronic control system is further configured to transport, using the pump device, a definable volume of the sample liquid, in the form of a liquid sample, along the fluid flow path into the sample container,
wherein the sampling apparatus concentrates or extracts the analyte present in the liquid sample;
an analytical instrument configured for the qualitative or quantitative determination of the at least one analyte; and
a transfer device configured to provide a sample of the at least one analyte, which is concentrated or extracted using the sampling apparatus.

39. The measurement system of claim 38, wherein the transfer device is configured to automatically transfer the sample of the concentrated or extracted analyte to the analytical instrument.

40. The measurement system of claim 38, further comprising an electronic control system configured to control the transfer device for the automatic transfer of a sample of the concentrated or extracted analyte to the analytical instrument.

41. The measurement system of claim 38, wherein the analytical instrument is designed to carry out the qualitative or quantitative determination of the analyte partially or completely automatically.

42. A method for the automatic provision of a sample for a qualitative or quantitative determination of at least one analyte in a sample liquid, comprising:
removing a definable volume of the sample liquid, in the form of a liquid sample, from the sampling station using an automatic sampling apparatus from the sample liquid present at a sampling station;
transporting the volume of the sample liquid, in the form of a liquid sample, into a sample container; and
bringing the liquid sample into contact with a collection matrix that immobilizes the analyte.

43. The method of claim 42, wherein the collection matrix is placed in the sample container or is arranged in a flow-through unit fluidically connected to the sample container.

44. The method of claim 42, further comprising:
removing the collection matrix with the analyte from the device or eluting the analyte from the collection matrix to form a solution of the analyte as a sample for subsequent qualitative or quantitative determination.

45. The method of claim 42, further comprising storing an identifier of the liquid sample and a time stamp representing the time the liquid sample was taken from the sampling station in an electronic control system of the automatic sampling apparatus.

46. The method of claim 45, further comprising acquiring at least one measured value of at least one measurand of the sample liquid using an additional sensor and storing the measured value in the electronic control system for identifying the liquid sample.

47. The method of claim 45, further comprising acquiring a temperature or a temperature profile to which the liquid sample is exposed from the time at which it is taken, and storing measured values reflecting the temperature or the temperature profile in the electronic control system for identifying the liquid sample.

48. The method of claim 45, further comprising transferring the identifier of the liquid sample, the associated time stamp, and further associated data or measured values to an operating device, to an analytical instrument used for the analysis of the provided sample, or to a server, which is connected to the analytical instrument or the electronic control system for communication and which stores the data or measured values obtained from the electronic control system or the analytical instrument, the memory of which server is accessed by using the non-transitory computer readable medium in order to display or to perform a further analysis of the data or measured values.

49. A method for the qualitative or quantitative determination of at least one analyte in a sample liquid, comprising:
providing the sample using the following steps:
removing a definable volume of the sample liquid, in the form of a liquid sample, from the sampling station using an automatic sampling apparatus from the sample liquid present at a sampling station;
transporting the volume of the sample liquid, in the form of a liquid sample, into a sample container; and
bringing the liquid sample into contact with a collection matrix that immobilizes the analyte; and
qualitatively or quantitatively determining the analyte using an immunological assay or a real-time, qPCR-based method.

50. The method of claim 49, further comprising:
determining a concentration of the analyte in the original liquid sample, with additional inclusion of at least one measured value acquired at the sampling station, wherein the at least one measured value is provided by an electronic control system of the sampling apparatus.

51. The method of claim 49, further comprising communicating determined analytical findings and additional data to a non-transitory computer readable medium.

52. The method of claim 51, wherein the non-transitory computer readable medium generates and displays a graphical representation of a spatial distribution or a temporal development of the concentration of the analyte at a plurality of sampling stations.
